(19) United States
(12) Patent Application Publication
(10) Pub. No.: US 2005/0070536 A1
(43) Pub. Date: Mar. 31, 2005

(54) Novel tablets and capsules and a process for its preparation

(76) Inventors: Thyge Borup Hjorth, Farum (DK); Breian Knudsen, Albertslund (DK)

Correspondence Address: NOVO NORDISK, INC.;PATENT DEPARTMENT, 100 COLLEGE ROAD WEST, PRINCETON, NJ, 08540, US

(21) Appl. No.: 10/985,719

(22) Filed: Nov. 10, 2004

Related U.S. Application Data
(63) Continuation of application No. 10/101,456, filed on Mar. 12, 2002.
(60) Provisional application No. 60/277,167, filed on Mar. 20, 2001.

(30) Foreign Application Priority Data
Mar. 12, 2001 (DK) PA 2001 00413

(51) Int. Cl. A61K 31/538; A61K 31/7012

(52) U.S. Cl. 514/229.8; 514/53

(57) ABSTRACT

Tablets and capsules can be prepared with improved homogenous distribution of the active ingredient if microcrystalline cellulose and silicon dioxide are used as some of the starting materials.

### CROSS-REFERENCE TO RELATED APPLICATIONS

This application is a continuation of application Ser. No. 10/101,456 filed on Mar. 12, 2002, and claims priority under 35 U.S.C. 119 of Danish application no. PA 2001 00413 filed on Mar. 12, 2001; of U.S. provisional application no. 60/277,167 filed on Mar. 20, 2001. and of application Ser. No. 10/101,456 filed on Mar. 12, 2002 in the U.S. is claimed under 35 U.S.C. 120, the contents of all of which are fully incorporated herein by reference.

### FIELD OF THE INVENTION

The present invention relates to novel tablets and capsules with relatively low amounts of the active ingredient. The present invention further relates to tablets and capsules comprising as the active ingredient (−) 3-[4-[2-(phenoxazin-10-yl) ethoxy]phenyl]-2-ethoxypropanoic acid or a pharmaceutically acceptable salt thereof.

### BACKGROUND OF THE INVENTION

In U.S. Pat. Nos. 5,948,438 and 6,106,865 oral solid dosage forms comprising microcrystalline cellulose and a compressibility augmenting agent such as silicon dioxide are described.

(2S)(−)-3-[4-[2-(phenoxazin-10-yl) ethoxy]phenyl]-2-ethoxypropanoic acid (also referred to herein as (−) 3-[4-[2-(phenoxazin-10-yl)ethoxy]phenyl]-2-ethoxypropanoic acid) and pharmaceutically acceptable salts thereof has been found useful in the treatment of type 2 diabetes acting as an insulin sensitizer as disclosed in e.g. PCT Publication WO 99/19313, WO 00/50414 and WO 00/63192, which are incorporated herein by reference. Pharmaceutical compositions containing (−) 3-[4-[2-(phenoxazin-10-yl)ethoxy]phenyl]-2-ethoxypropanoic acid as active ingredient or a pharmaceutically acceptable salt thereof are described in WO01/74363.

One aspect of the present invention is to provide tablets and capsules having improved homogeneity.

Another aspect of the present invention is to provide tablets and capsules having improved homogeneity with respect to the content of active ingredient.

Another aspect of the present invention is to provide tablets and capsules having improved homogeneity with respect to the distribution of active ingredient.

Another aspect of the present invention is to provide tablets and capsules having a low content of active ingredient.

### SUMMARY OF THE INVENTION

It has now been found that a tablet or capsule wherein the content of active ingredient in the tablet or capsule is between about 3% (weight/weight) and about 0.001% (weight/weight) can be prepared with improved homogenous distribution of the active ingredient if microcrystalline cellulose and silicon dioxide are used as some of the starting materials. Surprisingly, it has now been found that a tablet or capsule wherein the content of active ingredient in the tablet or capsule is not more than about 2% (weight/weight) can be prepared with improved homogenous distribution of the active ingredient if microcrystalline cellulose and silicon dioxide are used as some of the starting materials. Using these starting materials, it is even possible to prepare tablets and capsules wherein the content of active ingredient in the tablet or capsule is not more than about 1%, not more than about 0.5%, not more than about 0.05%, not more than about 0.01%, and even not more than about 0.005% (weight/weight).

A further aspect of the present invention provides a tablet or capsule with improved homogenous distribution which comprises microcrystalline cellulose and silicon dioxide and (−) 3-[4-[2-(phenoxazin-10-yl) ethoxy]phenyl]-2-ethoxypropanoic acid or a pharmaceutically acceptable salt thereof.

### DETAILED DESCRIPTION OF THE INVENTION

Generally, any sort of microcrystalline cellulose can be used for the preparation of the tablets and capsules according to the present invention. However, it is preferred that the microcrystalline cellulose used for the preparation of the tablets and capsules according to the present invention has a bulk density above 0.35 g/ml. Furthermore, it is preferred that the microcrystalline cellulose used for the preparation of the tablets and capsules according to the present invention has a particle size above about 50 micrometer. A tablet or capsule according to the present invention comprises preferably microcrystalline cellulose in an amount of at least 20%, more preferred of at least 30% and even more preferred of at least 40% (weight/weight) and most preferred in an amount of at least 45% (weight/weight). For example, the tablet or capsule according to the invention comprises microcrystalline cellulose in amounts ranging from 20% to 80% (weight/weight), amounts from 30% to 70% are preferred while amounts from 40% to 60% are even more preferred and amounts ranging from 45% to 55% are especially preferred.

Similarly, any sort of silicone dioxide can be used for the preparation of the tablets and capsules according to the present invention. However, it is preferred that the silicone dioxide used for the preparation of the tablets and capsules according to the present invention has a particle size from about 1 nanometer to about 100 micrometer. A tablet or capsule according to the present invention comprises preferably an amount of silicone dioxide in the range from 0.1 to 5%, preferably in the range from 0.2% to 3%, even more preferred in the range from 0.5% to 1,5% (weight/weight).

In another embodiment according to the present invention, the tablet or capsule further comprises mannitol. In a preferred embodiment according to the invention, the tablet or capsule comprises mannitol in an amount of at least 20%, preferably at least 30% an even more preferred at lest 40% (weight/weight) and most preferred in an amount of at least 45% (weight/weight). In preferred embodiments of the invention, the tablet or capsule comprises mannitol in amounts ranging from 20% to 80% (weight/weight), amounts from 30% to 70% are preferred while amounts from 40% to 60% are even more preferred and amounts ranging from 45% to 55% are especially preferred.

In another embodiment of the present invention, the tablet or capsule comprises mannitol and microcrystalline cellulose in proportions between 2:8 and 8:2, preferably between 3:5 and 5:3, more preferred between 4:6 and 6:4, and even more preferred between 45:55 and 55:45.

In another embodiment of the present invention, the tablet or capsule comprises talc in the range from 0.1 to 10% (weight/weight).

In a further preferred embodiment of the invention, the tablet or capsule comprises microcrystalline cellulose in an amount ranging from 45% to 55% and silicon dioxide in an amount from 0,5% to 1,5% (weight/weight).

In another embodiment of the invention, the tablet or capsule comprises: (i) an active ingredient selected from the group consisting of (−) 3-[4-[2-(phenoxazin-10-yl) ethoxy]phenyl]-2-ethoxypropanoic acid, pharmaceutically acceptable salt, ester, metabolite, hydrate, solvate, polymorph, and pro-drug form thereof, (ii) microcrystalline cellulose; and (iii) silicon dioxide, wherein the amount of said active ingredient is between about 0.01% and about 2,0% (weight/weight); the amount of said microcrystalline cellulose is between about 40% and about 50% (weight/weight); and the amount of said silicone dioxide is between about 0.8% and about 1.2% (weight/weight).

Non-limiting examples of a formulation according to invention include the following (expressed as % weight/total weight of each ingredient):

| | |
|---|---|
| Active ingredient | 1% |
| Microcrystalline cellulose | 97.8-98.2% |
| Silicon dioxide | 0.8-1.2% |
| Active ingredient | 1% |
| Microcrystalline cellulose | 47% |
| Silicon dioxide | 1% |
| Mannitol | 47% |
| Talc | 4% |
| Active ingredient | 1% |
| Microcrystalline cellulose | 45.2% |
| Silicon dioxide | 0.8% |
| Mannitol | 49% |
| Talc | 4% |
| Active ingredient | 0.5% |
| Microcrystalline cellulose | 98.3-98.7% |
| Silicon dioxide | 0.8-1.2% |
| Active ingredient | 2% |
| Microcrystalline cellulose | 96.8-97.2% |
| Silicon dioxide | 0.8-1.2% |
| Active ingredient | 0.2% |
| Microcrystalline cellulose | 98.6-99.0% |
| Silicon dioxide | 0.8-1.2 |
| Active ingredient | 2% |
| Microcrystalline cellulose | 46.5% |
| Silicon dioxide | 1% |
| Mannitol | 46.5% |
| Talc | 4 |

In a preferred embodiment of the present invention, the tablets or capsules are prepared using one of the commercial products marketed under the designations ProSolv HD® 90, ProSolv SMCC® 50, and ProSolv SMCC® 90 by the firm PenWest, each of which contain both microcrystalline cellulose and silicon dioxide.

In one aspect of the present invention, the tablets or capsules have a relative standard deviation (RSD) for the content of active ingredient in the tablet or capsule which is not more than about 4%. Preferably, the RSD value is not more than about 3%, more preferred the RSD value is not more than about 2,5%, and even more preferred the RSD value is not more than about 2%, especially preferred the RSD value is not more than about 1,5%, and even more preferred the RSD value is not more than about 1%.

The relative standard deviation (RSD) for the content of active ingredient in the tablets of the present invention can be determined as described in the United States Pharmacopoeia (USP 24) 2000, chapter 905 using the test for Uniformity of Dosage Units, <905>, USP XV.

The bulk and tapped density can be determined as described in method BTD-8, Handbook of Pharmaceutical Excipients, 2nd ed., 1994. Particle size distribution can be determined by Sieve analysis using US standard sieves.

The tablets and capsules according to the present invention can be prepared in a manner known per se. More specifically, the tablets and capsules may be prepared by conventional techniques, e.g. as described in Remington: The Science and Practice of Pharmacy, 19th edition, 1995. According to a preferred feature of the present invention, the tablet or capsule according to the present invention are prepared by direct compression. Direct compression has the advantage that it is possible to first mix all the ingredients which are to be present in the final tablet or capsule and then to subject the mixture to direct compression.

The active ingredient present in the tablets and capsules of the present invention can be any pharmaceutical such as analgesics and anti-inflammatory agents, anthelminthics, anti-arrhythmic agents, antibacterial agents, anticoagulants, anti-depressants, antidiabetic agents, antiepileptics, antifungal agents, antigout agents, antihypertensive agents, antimalarials, antimigraine agents, antimuscarinic agents, antineoplastic agents, immunosuppressants, antiprotozoal agents, antithyroid agents, antiviral agents, anxiolytic sedatives, hypnotics, neuroleptics, beta-adrenoceptor blocking agents, calcium regulating agents, cardiac inotropic agents, chelating agents, antidotes, antagonists, corticosteroids, diuretics, dopaminergic antiparkinsonian agents, gastro-intestinal agents, anaesthetics, hormones, lipid regulating agents, anti-angina agents, vitamins, anti-asthma agents, skeletal muscle relaxants, stimulants, anoretics, sympatomimetics, thrombolytic agents, and vasodilators.

An example of a specific medicaments is (−) 3-[4-[2-(phenoxazin-10-yl)ethoxy]phenyl]-2-ethoxypropanoic acid and pharmaceutically acceptable salts thereof. Pharmaceutically acceptable salts forming part of this invention include salts such as alkali metal salts like Li, Na, and K salts, alkaline earth metal salts like Ca and Mg salts, salts of organic bases such as lysine, arginine, guanidine, diethanolamine, choline and the like, ammonium or substituted ammonium salts, aluminium salts. Salts may include acid addition salts where appropriate which are, sulphates, nitrates, phosphates, perchlorates, borates, hydrohalides, acetates, tartarates, maleates, citrates, succinates, palmoates, methanesulplionates, benzoates, salicylates, hydroxynaphthoates, benzenesulfonates, ascorbates, glycerophosphates, ketoglutarates and the like. Further examples of pharmaceutically acceptable inorganic or organic acid addition salts include the pharmaceutically acceptable salts listed in J. Pharm. Sci. 1977, 66, 2, which is incorporated herein by reference.

The present invention also encompasses esters, metabolites, hydrates, solvates, polymorphs, and pro-drug forms of (−) 3-[4-[2-(phenoxazin-10-yl)ethoxy]phenyl]-2-ethoxypropanoic acid.

In a preferred embodiment, (−) 3-[4-[2-(phenoxazin-10-yl)ethoxy]phenyl]-2-ethoxypropanoic acid, arginine is used in the present invention.

Another example of a specific medicament is 5-[[4-[3-Methyl-4-oxo-3,4-dihydro-2-quinazolinyl]methoxy]phenyl-methyl]thiazolidine-2,4-dione and pharmaceutically acceptable salts thereof as active ingredient as described in PCT Publication WO 97/41097. The present invention also encompasses esters, metabolites, hydrates, solvates, polymorphs, and prodrug forms of 5-[[4-[3-Methyl-4-oxo-3,4-dihydro-2-quinazolinyl]methoxy]phenyl-methyl]thiazolidine-2,4-dione.

Examples of suitable carriers present in the tablets and capsules according to the present invention are water, salt solutions, alcohols, polyethylene glycols, polyhydroxyethoxylated castor oil, peanut oil, olive oil, gelatine, lactose, terra alba, sucrose, mannitol, sorbitol, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The tablets and capsules may also include wetting agents, emulsifying and suspending agents, preserving agents, sweetening agents or flavouring agents. The tablets and capsules of the invention may be formulated so as to provide quick, sustained, or delayed release of the active ingredient after administration to the patient by employing procedures well known in the art.

The tablets and capsules can be sterilized and mixed, if desired, with auxiliary agents, emulsifiers, salt for influencing osmotic pressure, buffers and/or colouring substances and the like, which do not deleteriously react with the active compounds.

If a solid carrier is used for oral administration, the preparation may be tabletted, placed in a hard gelatin capsule in powder or pellet form or it can be in the form of a troche or lozenge.

Tablets, dragees, or capsules having talc and/or a carbohydrate carrier or binder or the like are particularly suitable for oral application. Preferable carriers for tablets, dragees, or capsules include lactose, corn starch, and/or potato starch.

The tablets and capsules of the invention may be administered to a mammal, especially a human in need of such treatment, prevention, elimination, alleviation or amelioration of various diseases as mentioned above. Such mammals include also animals, both domestic animals, e.g. household pets, and non-domestic animals such as wildlife.

The exact dosage of the tablets and capsules will depend upon the mode of administration, on the therapy desired, form in which administered, the subject to be treated and the body weight of the subject to be treated, and the preference and experience of the physician or veterinarian in charge.

The present invention will further be illustrated with the following non-exhaustive examples.

### EXAMPLE 1

| | |
|---|---|
| (−)3-[4-[2-(Phenoxazin-10-yl)ethoxy]phenyl]-2- | 3.860 g |
| ethoxypropanoic acid, arginine salt | |
| Microcrystalline cellulose | 1423 g |
| with 2% silicon dioxide | |
| Mannitol | 1423 g |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine salt, and 200g of microcrystalline cellulose with silicon dioxide is mixed in a drum mixer for 5 minutes. Mannitol and the rest of microcrystalline cellulose with silicon dioxide is added and mixing is continued for 10 minutes. Magnesium stearate and talc are added and mixed for further 5 minutes.

The powder mixture is compressed into tablets on a tabletting machine. The tablet weight is 110 mg.

The results from test for content uniformity according to USP gave the following results: Mean content of (−) 3-[4-[2-(phenoxazin-10-yl)ethoxy]phenyl]-2-ethoxypropanoic acid in each tablet:

0.0973 mg

RSD: 1.17%

### EXAMPLE 2

| | |
|---|---|
| (−)3-[4-[2-(Phenoxazin-10-yl)ethoxy]phenyl]-2- | 3.860 g |
| ethoxypropanoic acid, arginine salt | |
| Microcrystalline cellulose | 2846 g |
| with 2% silicon dioxide | |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine salt, and 200g of microcrystalline cellulose with silicon dioxide is mixed in a drum mixer for 5 minutes. The rest-of microcrystalline cellulose with silicon dioxide is added and mixing is continued for 10 minutes. Magnesium stearate and talc are added and mixed for further 5 minutes.

The powder mixture is compressed into tablets on a tabletting machine. The tablet weight is 110 mg.

The results from test for content uniformity according to USP gave the following results:

Mean content of (−) 3-[4-[2-(phenoxazin-10-yl)ethoxy]phenyl]-2-ethoxypropanoic acid in each tablet:

0.103 mg

RSD: 1.85%

### EXAMPLE 3

| | |
|---|---|
| (−)3-[4-[2-(Phenoxazin-10-yl)ethoxy]phenyl]-2- | 3.860 g |
| ethoxypropanoic acid, arginine salt | |
| Microcrystalline cellulose | 1708 g |
| with 2% silicon dioxide | |
| Mannitol | 1138 g |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine salt, and 200g of microcrystalline cellulose with silicon dioxide is mixed in a drum mixer for 5 minutes. Mannitol and the rest of microcrystalline cellulose with silicon dioxide is added and mixing is continued for 10 minutes. Magnesium stearate and talc are added and mixed for further 5 minutes.

The powder mixture is compressed into tablets on a tabletting machine. The tablet weight is 110 mg.

### EXAMPLE 4

| | |
|---|---|
| (−)3-[4-[2-(Phenoxazin-10-yl)ethoxy]phenyl]-2- | 3.860 g |
| ethoxypropanoic acid, arginine salt | |
| Microcrystalline cellulose | 1138 g |
| with 2% silicon dioxide | |
| Mannitol | 1708 g |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine salt, and 200g of microcrystalline cellulose with silicon dioxide is mixed in a drum mixer for 5 minutes. Mannitol and the rest of microcrystalline cellulose with silicon dioxide is added and mixing is continued for 10 minutes. Magnesium stearate and talc are added and mixed for further 5 minutes.

The powder mixture is compressed into tablets on a tabletting machine. The tablet weight is 110 mg.

### EXAMPLE 5

| | |
|---|---|
| (−)3-[4-[2-(Phenoxazin-10-yl)ethoxy]phenyl]-2- | 3.860 g |
| ethoxypropanoic acid, arginine salt | |
| Microcrystalline cellulose | 1897 g |
| with 2% silicon dioxide | |
| Mannitol | 949 g |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine salt, and 200g of microcrystalline cellulose with silicon dioxide is mixed in a drum mixer for 5 minutes. Mannitol and the rest of microcrystalline cellulose with silicon dioxide is added and mixing is continued for 10 minutes. Magnesium stearate and talc are added and mixed for further 5 minutes.

The powder mixture is compressed into tablets on a tabletting machine. The tablet weight is 110 mg.

### EXAMPLE 6

| | |
|---|---|
| (−)3-[4-[2-(Phenoxazin-10-yl)ethoxy]phenyl]-2- | 3.860 g |
| ethoxypropanoic acid, arginine salt | |
| Microcrystalline cellulose | 949 g |
| with 2% silicon dioxide | |
| Mannitol | 1897 g |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine salt, and 200g of microcrystalline cellulose with silicon dioxide is mixed in a drum mixer for 5 minutes. Mannitol and the rest of microcrystalline cellulose with silicon dioxide is added and mixing is continued for 10 minutes. Magnesium stearate and talc are added and mixed for further 5 minutes.

The powder mixture is compressed into tablets on a tabletting machine. The tablet weight is 110 mg.

### EXAMPLE 7

| | |
|---|---|
| (−)3-[4-[2-(Phenoxazin-10-yl)ethoxy]phenyl]-2- | 3.860 g |
| ethoxypropanoic acid, arginine salt | |
| Microcrystalline cellulose | 407 g |
| with 2% silicon dioxide | |
| Mannitol | 2439 g |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine salt, and 200g of microcrystalline cellulose with silicon dioxide is mixed in a drum mixer for 5 minutes. Mannitol and the rest of microcrystalline cellulose with silicon dioxide is added and mixing is continued for 10 minutes. Magnesium stearate and talc are added and mixed for further 5 minutes.

The powder mixture is compressed into tablets on a tabletting machine. The tablet weight is 110 mg.

### EXAMPLE 8

| | |
|---|---|
| (−)3-[4-[2-(Phenoxazin-10-yl)ethoxy]phenyl]-2- | 3.860 g |
| ethoxypropanoic acid, arginine salt | |
| Microcrystalline cellulose | 2439 g |
| with 2% silicon dioxide | |
| Mannitol | 407 g |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine salt, and 200g of microcrystalline cellulose with silicon dioxide is mixed in a drum mixer for 5 minutes. Mannitol and the rest of microcrystalline cellulose with silicon dioxide is added and mixing is continued for 10 minutes. Magnesium stearate and talc are added and mixed for further 5 minutes.

The powder mixture is compressed into tablets on a tabletting machine. The tablet weight is 110 mg.

### EXAMPLE 9

| | |
|---|---|
| (−)3-[4-[2-(Phenoxazin-10-yl)ethoxy]phenyl]-2- | 3.860 g |
| ethoxypropanoic acid, arginine salt | |
| Microcrystalline cellulose | 90 g |
| Lactose, monohydrate | 2885 |
| Silica, colloidal anhydrous | 6 g |
| Magnesium stearate | 15 g |

Manufacture:

(−) 3-[4-[2-(Phenoxazin-10-yl)ethoxy]phenyl]-2-ethoxypropanoic acid, arginine salt, and 90 g of microcrystalline cellulose is mixed in a drum mixer for 5 minutes. Lactose and silica is added and mixing is continued for 10 minutes. Magnesium stearate is added and mixed for further 5 minutes.

The powder mixture is compressed into tablets on a tabletting machine. The tablet weight is 110 mg.

### EXAMPLE 10

| | |
|---|---|
| (−)3-[4-[2-(Phenoxazin-10-yl)ethoxy]phenyl]-2- | 19.30 g |
| ethoxypropanoic acid, arginine salt | |
| Microcrystalline cellulose | 1693 g |
| with 2% silicon dioxide | |
| Mannitol | 1138 g |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine-salt, and 200g of microcrystalline cellulose with silicon dioxide is mixed in a drum mixer for 5 minutes. Mannitol and the rest of microcrystalline cellulose with silicon dioxide is added and mixing is continued for 10 minutes. Magnesium stearate and talc are added and mixed for further 5 minutes.

The powder mixture are compressed into tablets on a tabletting machine. The tablet weight is 110 mg.

### EXAMPLE 11

| | |
|---|---|
| (−)3-[4-[2-(Phenoxazin-10-yl)ethoxy]phenyl]-2- | 1.930 g |
| ethoxypropanoic acid, arginine salt | |
| Microcrystalline cellulose | 950 g |
| with 2% silicon dioxide | |
| Mannitol | 1898 g |
| Magnesium stearate | 30 g |
| Talc | 120 g |

Manufacture:

(−) 3-[4-[2-(Phenoxazin-10-yl)ethoxy]phenyl]-2-ethoxypropanoic acid, arginine salt, and 200g of microcrystalline cellulose with silicon dioxide is mixed in a drum mixer for 5 minutes. Mannitol and the rest of microcrystalline cellulose with silicon dioxide is added and mixing is continued for 10 minutes. Magnesium stearate and talc are added and mixed for further 5 minutes.

The powder mixture is compressed into tablets on a tabletting machine. The tablet weight is 110 mg.

1. A pharmaceutical composition comprising (i) (−) 3-[4-[2-(phenoxazin-10-yl) ethoxy]phenyl]-2-ethoxypropanoic acid or a pharmaceutically acceptable salt or ester, metabolite, hydrate, solvate, polymorph or prodrug form thereof as an active ingredient, (ii) microcrystalline cellulose, and (iii) silicone dioxide.

2. The pharmaceutical composition according to claim 1, wherein the pharmaceutically acceptable salt of (−) 3-[4-[2-(Phenoxazin-10-yl)ethoxy]phenyl]-2-ethoxypropanoic acid is the arginine salt.

3. The pharmaceutical formulation according to claim 1, wherein (i) the amount of said microcrystalline cellulose is between about 40% and about 50% (weight/weight); and (ii) the amount of said silicone dioxide is between about 0.8% and about 1.2% (weight/weight).

4. The pharmaceutical composition according to claim 1, further comprising mannitol.

5. The pharmaceutical composition according to claim 1, further comprising lactose.

6. The pharmaceutical composition according to claim 4, wherein the ratio of mannitol to microcrystalline cellulose is between 2:8 and 8:2.

7. The pharmaceutical composition according to claim 4, wherein the ratio of mannitol to microcrystalline cellulose is between 3:5 and 5:3.

8. The pharmaceutical composition according to claim 4, wherein the ratio of mannitol to microcrystalline cellulose is between 4:6 and 6:4.

9. The pharmaceutical composition according to claim 4, wherein the ratio of mannitol to microcrystalline cellulose is between 45:55 and 55:45.

10. The pharmaceutical composition according to claim 1, wherein the content of silicon dioxide is about 0.1 to about 5%.

11. The pharmaceutical composition according to claim 1, wherein the content of silicone dioxide is about 0.2% to about 3%.

12. The pharmaceutical composition according to claim 1, wherein the content of silicone dioxide is about 0.5% to about 1.5%.

13. The pharmaceutical composition according to claim 3, wherein (i) the amount of said microcrystalline cellulose is from about 45% to about 55% (weight/weight); and (ii) the amount of said silicone dioxide is from about 0,5% to 1,5% (weight/weight).

14. The pharmaceutical composition according to claim 1, wherein the microcrystalline cellulose has a bulk density above 0.35 g/ml.

15. The pharmaceutical composition according to claim 1, wherein the microcrystalline cellulose has a particle size above about 50 micrometer.

16. The pharmaceutical composition according to claim 1, wherein the silicone dioxide has a particle size from about 1 nanometer to about 100 micrometer.

17. The pharmaceutical composition according to claim 1, further comprising ProSolv HD® 90, ProSolv SMCC® 50, and ProSolv SMCC® 90.

18. The pharmaceutical composition according to claim 1, wherein the relative standard deviation (RSD) for the content of active ingredient in a tablet or capsule is not more than about 4%.

19. The pharmaceutical composition according to claim 1, wherein the relative standard deviation (RSD) value is not more than about 3%.

20. The pharmaceutical composition according to claim 1, wherein the relative standard deviation (RSD) value is not more than about 2.5%.

21. The pharmaceutical composition according to claim 1, wherein the relative standard deviation (RSD) value is not more than about 2%.

22. The pharmaceutical composition according to claim 1, wherein the relative standard deviation (RSD) value is not more than about 1,5%.

23. The pharmaceutical composition according to claim 1, wherein the relative standard deviation (RSD) value is not more than about 1%.

24. A pharmaceutical composition comprising (i) 5-[[4-[3-Methyl-4-oxo-3,4-dihydro-2-quinazolinyl]methoxy]phenyl-methyl]thiazolidine-2,4-dione or a pharmaceutically acceptable salt or ester, metabolite, hydrate, solvate, polymorph or prodrug form thereof as an active ingredient, microcrystalline cellulose and silicon dioxide.

25. The pharmaceutical formulation according to claim 24, wherein (i) the amount of said microcrystalline cellulose is between about 40% and about 50% (weight/weight); and (ii) the amount of said silicone dioxide is between about 0.8% and about 1.2% (weight/weight).

26. The pharmaceutical composition according to claim 24, further comprising mannitol.

27. The pharmaceutical composition according to claim 24, further comprising lactose.

28. The pharmaceutical composition according to claim 26, wherein the ratio of mannitol to microcrystalline cellulose is between 2:8 and 8:2.

29. The pharmaceutical composition according to claim 26, wherein the ratio of mannitol to microcrystalline cellulose is between 3:5 and 5:3.

30. The pharmaceutical composition according to claim 26, wherein the ratio of mannitol to microcrystalline cellulose is between 4:6 and 6:4.

31. The pharmaceutical composition according to claim 26, wherein the ratio of mannitol to microcrystalline cellulose is between 45:55 and 55:45.

32. The pharmaceutical composition according to claim 24, wherein the content of silicon dioxide is about 0.1 to about 5%.

33. The pharmaceutical composition according to claim 24, wherein the content of silicone dioxide is about 0.2% to about 3%.

34. The pharmaceutical composition according to claim 24, wherein the content of silicone dioxide is about 0.5% to about 1.5%.

35. The pharmaceutical composition according to claim 24, wherein (i) the amount of said microcrystalline cellulose is from about 45% to about 55% (weight/weight); and (ii) the amount of said silicone dioxide is from about 0,5% to 1,5% (weight/weight).

36. The pharmaceutical composition according to claim 24, wherein the microcrystalline cellulose has a bulk density above 0.35 g/ml.

37. The pharmaceutical composition according to claim 24, wherein the microcrystalline cellulose has a particle size above about 50 micrometer.

38. The pharmaceutical composition according to claim 24, wherein the silicone dioxide has a particle size from about 1 nanometer to about 100 micrometer.

39. The pharmaceutical composition according to claim 24, further comprising ProSolv HD® 90, ProSolv SMCC® 50, and ProSolv SMCC® 90.

40. The pharmaceutical composition according to claim 24, wherein the relative standard deviation (RSD) for the content of active ingredient in a tablet or capsule is not more than about 4%.

41. The pharmaceutical composition according to claim 24, wherein the relative standard deviation (RSD) value is not more than about 3%.

42. The pharmaceutical composition according to claim 24, wherein the relative standard deviation (RSD) value is not more than about 2.5%.

43. The pharmaceutical composition according to claim 24, wherein the relative standard deviation (RSD) value is not more than about 2%.

44. The pharmaceutical composition according to claim 24, wherein the relative standard deviation (RSD) value is not more than about 1,5%.

45. The pharmaceutical composition according to claim 24, wherein the relative standard deviation (RSD) value is not more than about 1%.
